(19) United States
(12) Patent Application Publication
(10) Pub. No.: US 2022/0168044 A1
(43) Pub. Date: Jun. 2, 2022

(54) Augmented Reality Dental Display System and Method

(71) Applicant: August de Oliveira, Encino, CA (US)

(72) Inventor: August de Oliveira, Encino, CA (US)

(21) Appl. No.: 17/108,949

(22) Filed: Dec. 1, 2020

(51) Int. Cl. A61B 34/10; G06T 7/00; G06T 19/00; G06T 17/00

(52) U.S. Cl. CPC A61B 34/10; G06T 7/0012; A61B 2034/107; G06T 17/00; A61B 2034/105; G06T 19/006

(57) ABSTRACT

An augmented reality dental display system includes a first database containing a 3D intra oral image, a second database containing a 3D oral anatomy image, one or more tooth markers configured for applying on one or more of the patient's teeth for tooth identification, and a processor combining the 3D intra oral image and the 3D oral anatomy image to form an augmented reality displayable image. When the augmented reality headset is worn, the tooth marker is read by the augmented reality headset to execute the processor for visually superimposing the augmented reality displayable image on the patient's teeth during a dental surgery.

### BACKGROUND

### 1. Field of the Invention

The present invention primarily relates to a dental imaging system. More particularly, the present invention relates to an augmented reality dental display system and method to display 3D oral anatomy images via an augmented reality headset.

### 2. Discussion of the Related Art

Like medical radiographs, dental radiographs allow dentist to accurately evaluate, diagnose and treat different dental problems of a patient. Accordingly, there are two common types of diagnostic radiographs in dental offices, which are X-ray radiography and intraoral imaging device.

Dental X-rays are one of the great diagnostic tools to capture anatomy images that can help the dentists to diagnose any cavities, tooth decay or gum disease which are not visible to the naked eye. One type of X-ray radiography is intraoral radiography, wherein radiographic images of few teeth are captured at a time by inserting a small film cards in the patient's mouth. An advanced X-ray radiography is cephalometric x-ray that can capture a radiographic image of the entire head of the patient especially for diagnosing misalignment of the jaw and bite problems.

One type of the intraoral imaging device is an intraoral camera for capturing images of teeth and their supporting structures to view healthy areas and/or diseased sites in the patient's mouth. Another type of the intraoral imaging device is an intraoral scanner is a device to capture a direct optical impression. These intraoral imaging devices are widely used for dental restorations, such as crowns, veneers and implant frameworks.

These diagnostic radiographs, which are X-ray radiography and intraoral imaging device, can only be considered as a supplemental information. During dental surgery, such as implants or nerve extraction, based on these radiographic images, the success or failure of the surgery will totally depend on the surgeon's experience to determine where the nerves and bones are and how deep the drilling hole is formed.

Accordingly, it would be highly desirable to develop an overlaying visual information for the surgery in order to enhance the accuracy and efficacy of the surgery and the surgery's experience during surgery, so as to significantly reduce the surgery time and increase the success rate of the surgery.

### BRIEF SUMMARY OF THE INVENTION

According to the present invention, an augmented reality dental display system and method is described. The augmented reality dental display system and its method to configured to display 3D oral anatomy images via an augmented reality headset.

According to the present invention, the augmented reality dental display system comprises a first database containing a 3D intra oral image, a second database containing a 3D oral anatomy image, one or more tooth markers configured for applying on one or more of the patient's teeth for tooth identification, and a processor combining the 3D intra oral image with the 3D oral anatomy image to form an augmented reality displayable image. When the augmented reality headset is worn, the tooth marker is read by the augmented reality headset to execute the processor for visually superimposing the augmented reality displayable image on the patient's teeth during a dental surgery.

The present invention further provides a method of overlaying teeth anatomy on a patient, which comprises the following steps.

(A) Store a 3D intra oral image in a first database.

(B) Store a 3D oral anatomy image in a second database.

(C) Form an augmented reality displayable image by combining the 3D intra oral image with the 3D oral anatomy image via a processor.

(D) Apply one or more tooth markers on one or more of the patient's teeth for tooth identification, wherein the tooth marker is a readable code for an augmented reality headset.

(E) Visually superimpose the augmented reality displayable image on the patient's teeth during a dental surgery when the tooth marker is read by the augmented reality headset.

It is another object of the present invention to provide an augmented reality dental display system and method, wherein one or more computer readable codes embedded in one or more of the 3D teeth crown images of the 3D intra oral image to retrieve the augmented reality displayable image on the patient's teeth when the tooth marker is read by the augmented reality headset.

It is another object of the present invention to provide an augmented reality dental display system and method, wherein different images can be switched, individual and/or combined for being superimposed on the patient's tooth.

It is another object of the present invention to provide an augmented reality dental display system and method, wherein the operator, such as a surgeon or a dentist, is able to know where the nerve and bone are, and know how deep the hole can be drilled at the patient's tooth by visually superimposing the augmented reality displayable image on the patient's teeth.

It is another object of the present invention to provide an augmented reality dental display system and method, which enable the operator to quickly and easily recognize the intraoral anatomy image of the patient so as to help the operator to timely and precisely complete the dental surgery.

It is another object of the present invention to provide an augmented reality dental display system and method, which is easy to use without assistance, and inexpensive to operate.

It is another object of the present invention to provide an augmented reality dental display system and method, wherein the method thereof is simply and effective to form augmented reality displayable image while being time effective.

For a more complete understanding of the present invention with its objectives and distinctive features and advantages, reference is now made to the following specification and to the accompanying drawings.

### BRIEF DESCRIPTION OF THE DRAWING(S)

These and other features, aspects and advantages of the present invention will become better understood from the following description, appended claims, and accompanying figures where:

FIG. 1 is a block diagram illustrating augmented reality dental display system according to an embodiment of the present invention.

FIG. 2 illustrates a 3D intra oral image according to the embodiment of the present invention.

FIG. 3 illustrates a 3D oral anatomy image according to the embodiment of the present invention.

FIG. 4 illustrates the tooth marker applied on the patient's teeth according to the embodiment of the present invention.

FIG. 5 is a flow diagram illustrating augmented reality dental display method according to the embodiment of the present invention.

### DETAILED DESCRIPTION OF THE INVENTION

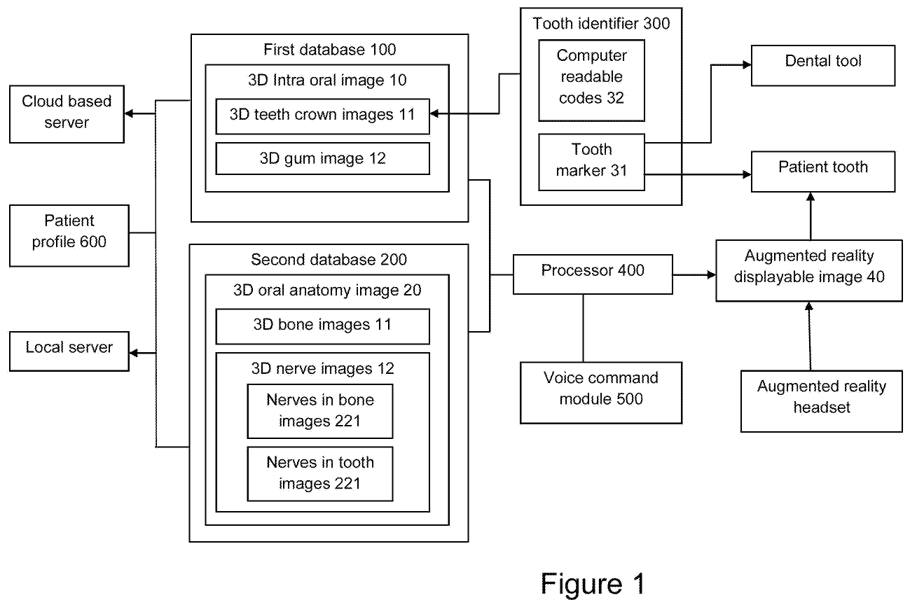

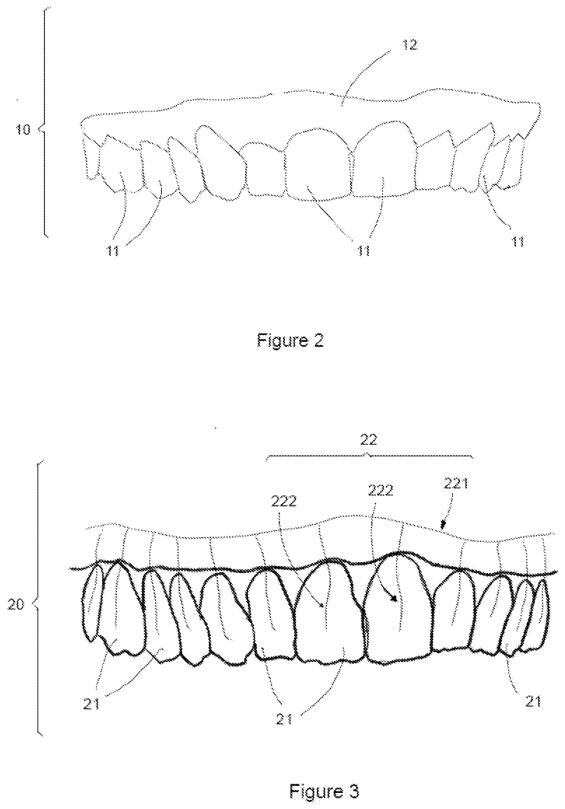

For purposes of description herein the terms “upper,” “lower,” “right,” “left,” “rear,” “front,” “vertical,” “horizontal” and derivatives thereof shall relate to the invention as oriented in FIGS. 1 and 2. However, it is to be understood that the invention may assume various alternative orientations and step sequences, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

As used in this disclosure, the term “comprise” and variations of the term, such as “comprising” and “comprises,” are not intended to exclude other additives, components, integers or steps.

It should be appreciated that the terms “first”, “second”, “one”, “a”, and “an” in the following description refer to “at least one” or “one or more” in the embodiment. In particular, the term “a” in one embodiment may refer to “one” while in another embodiment may refer to “more than one”. Therefore, the above terms should not be an actual numerical limitation of the elements of the present invention.

It should be appreciated that the terms “and” and “or” in the following description may refer to “one” or “more than one” in the accompanying drawings for easy understanding of the present invention without limiting the actual number of the present invention. Therefore, the above terms should not be an actual numerical limitation of the elements of the present invention.

It should be appreciated that the terms “first” and “second” in the following description may refer to different areas, regions, or modules, and the terms “first” and “second” should not be actual sequence limitation or should not present different types of the present invention.

In order to explain the technical solution of the present invention, the following description is made through specific embodiments.

FIGS. 1 to 3 illustrate augmented reality dental display system incorporating with an augmented reality (AR) headset for assisting an operator, such as a surgeon, doctor, or a dentist, to perform a dental surgery. According to a preferred embodiment, the augmented reality dental display system comprises a first database 100, a second database 200, a tooth identifier 300 and a processor 400. The first database 100, the second database 200 and the processor 400 can be combined to form a computer for image editing and modification.

In one embodiment, the first database 100 is configured to contain a 3D intra oral image 10 which is constructed to have a plurality of 3D teeth crown images 11 for showing crown portions of patient's teeth and a 3D gum image 12 for showing a gum of the patient. FIG. 2 shows an image of an upper jaw as the 3D intra oral image 10, wherein images of crown portions of the upper teeth are shown as the 3D teeth crown images 11, and an image of an upper gum is shown as the 3D gum image 12.

It is preferred that the 3D intra oral image 10 is captured by an intra oral scanner to record the crown portions of the patient's teeth and to obtain a 3D visual crown information thereof. For example, the intra oral scanner can be “CEREC” or “Trios”.

The second database 200 is configured to contain a 3D oral anatomy image 20 which is constructed to have a plurality of 3D bone images 21 for showing a jaw bone and root portions of the teeth of the patient and a plurality of 3D nerve images 22 for showing nerves of the patient. FIG. 3 shows an image of the upper jaw as the 3D oral anatomy image 20, wherein images of upper jaw bone and root portions of the upper teeth are shown is shown as the 3D bone images 21, and images of the nerves are shown as the 3D nerve images 22. Further, the 3D nerve images 22 contain nerves in bone images 221 and nerves in tooth images 222.

It is preferred that the 3D oral anatomy image 20 is captured by dental CT scanner to record bone and nerve anatomy images with nerve in bone and nerve in tooth images. For example, patient is scanned in the dental CT scanner (CBCT) and the image files (DICOM) are exported into the existing software, such as “Blue Sky Plan” and the nerve anatomy is marked either on nerves in bone or nerves in teeth. Software that makes crowns, such as “ExoCAD” or “3Shape Design Studio”, can also be used if the doctor wants to know the amount of tooth to be drilled by designing the crown in the software and then by using a tool of “Boolean Subtraction”, the shape that the doctor needs to cut on the tooth to get a good crown can then be superimposed in the patient's mouth using the AR headset.

According to the present invention, the processor 400 is configured to combine the 3D intra oral image 10 and the 3D oral anatomy image 20 to form an augmented reality displayable image 40 for visually superimposing on the patient's teeth during a dental surgery. In one embodiment, the first database 100 and the second database 200 are remotely stored in a cloud based server, wherein all the images can be remotely retrieved from the cloud based server. Alternatively, the first database 100 and the second database 200 can be stored in a local server.

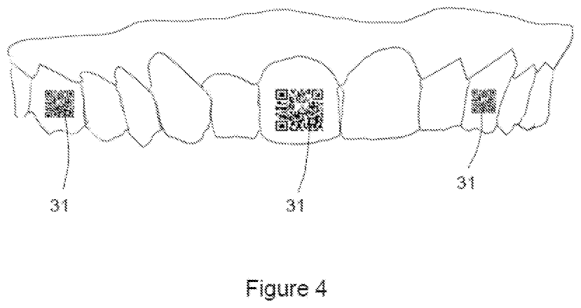

The tooth identifier 300 comprises one or more tooth marker 31 configured for applying on one or more of the patient's teeth for tooth identification. As shown in FIG. 4, two tooth marker 31 are applied on an exterior surfaces (crown surfaces) of the teeth respectively. In one embodiment, the tooth marker 31 is a fluorescence dye adapted for directly applying on the exterior surface of the patient's tooth. For example, the tooth marker 31 can be constructed to have a sticker film, wherein the fluorescence dye is applied on the sticker film. Therefore, by overlapping the sticker film on the exterior surface of the tooth, the fluorescence dye of the tooth marker 31 can be precisely applied on the tooth, then the sticker film can be removed from the tooth to remain the fluorescence dye of the tooth marker 31 thereon. In another embodiment, the sticker film can be made of biodegradable material directly sticking and staying on the exterior surface of the tooth to keep the fluorescence dye of the tooth marker 31 thereon.

It is preferred that the tooth marker 31 is a visual, machine-readable form. In one example, the tooth marker 31 is in form of a QR code marker adapted for directly applying on the exterior surface of the patient's tooth. Alternatively, the tooth marker 31 can be in form of barcode.

It is worth mentioning that the 3D intra oral image 10 can be embodied as the tooth marker 31 because the 3D intra oral image 10 contains dental features of the patient such as tooth morphology, variations in shape and size, restorations, missing tooth, wear patterns, teeth relative location configuration, color and position of the tooth, and other peculiar dental anomalies. These dental features will give every individual a unique identity. Therefore, no physical tooth marker 31 is applied on the patient's tooth as the 3D intra oral image 10 represents the tooth marker 31 for being read by the augmented reality headset.

It is appreciated that the tooth marker 31 can also be applied on any dental tool for being read by the augmented reality headset. The dental tool can be a dental handpiece or a dental drill. Take the dental drill as an example, the tooth marker 31 can be in form of a QR code marker applied on the surface of the dental drill, so that when the tooth marker 31 is read by the augmented reality headset to identify the tool as the dental drill, the operator is able to determine the length of drilling in bone as well as the length in teeth and root canals with rotary files. These drill bits and files would have a known length from the dental drill and it would appear in the AR display. In other words, information of the dental tool is pre-stored in the tooth marker 31, wherein when the tooth maker 31 on the dental tool is read, the augmented reality image of the dental tool will be visually incorporated with the augmented reality displayable image 40 during the dental surgery.

According to the present invention, the tooth identifier 300 further comprises one or more computer readable codes 32 embedded in the 3D intra oral image 10 and assigned to one or more of the tooth markers 31. In one example, the computer readable code 32 is embedded in the 3D teeth crown image 11 of the 3D intra oral image 10. When the tooth marker 31 is read via the augmented reality headset, the augmented reality displayable image 400 is retrieved for visually superimposing on the patient's teeth. In one example, the computer readable code 32 is a QR code being set corresponding to the QR code marker of the tooth marker 31. In another example, the computer readable code 32 is a barcode being set corresponding to the barcode marker of the tooth marker 31.

The image processing software is executed by the processor 400 to combine the 3D intra oral image 10 with the 3D oral anatomy image 20 to form the augmented reality displayable image 40, so that the augmented reality displayable image 40 is retrieved for visually superimposing on the patient's teeth during the dental surgery when the augmented reality headset is worn.

According to the present invention, any one or more of the images can be retrieved as the augmented reality displayable image 40 to be visually superimposed on the patient's teeth. Particularly, one or more of the 3D teeth crown image 11, the 3D gum image 12, the 3D bone image 21, and the 3D nerve image 22 are selected as the augmented reality displayable image 40 for visually superimposing on the patient's tooth.

When the operator wears the augmented reality headset to read the tooth marker 31 on the patient tooth during the dental surgery, one of the images can be retrieved to visually superimpose on the patient's tooth. Further, the augmented reality displayable image 40 can be switched from one image to another image. For example, the 3D gum image 12 as the augmented reality displayable image 40 can be initially displayed and can be switched to a combination of the nerves in bone images 221 and nerves in tooth images 222 as the augmented reality displayable image 40.

A patient profile 600 is provided for storing patient personal information and linking to the corresponding 3D intra oral image 10 and 3D oral anatomy image 20 in the first database 100 and the second database 200 respectively. Accordingly, the patient profile 600 is automatically retrieved to display the augmented reality displayable image 40 when the tooth marker 31 is a read by the augmented reality headset. For example, one patient profile 600 is assigned to the patient A, wherein the corresponding 3D intra oral image 10 and 3D oral anatomy image 20 of the patient A is linked to the patient profile 600, so that when the tooth marker 31 set for the patient A is read, the corresponding augmented reality displayable image 40 will be automatically retrieved.

According to the present invention, the processor 400 is further configured to modify the augmented reality displayable image 40 to change colors, to zoom in-and-out, to 3-dimensional rotate thereof. When the augmented reality headset is worn, the operator is able to modify the augmented reality displayable image 40 by his or her hands and/or fingers. For example, a particular 3D teeth crown image 11 with the corresponding nerves in bone image 221 and nerves in tooth image 222 can be enlarged and rotated in 3 dimensional directions, so that the operator is able to precisely know the length of the tooth and location of the nerves.

As shown in FIG. 1, the augmented reality dental display system further comprising a voice command module 500 operatively linked to the processor 400, wherein the operator is able to voice control the processor 400 when the augmented reality headset is worn. Accordingly, in response to a voice input from the augmented reality headset to the voice command module 500, the processor 400 is activated to selectively retrieve one or more of the 3D teeth crown image 11, the 3D gum image 12, the 3D bone image 21, and the 3D nerve image 22 as the augmented reality displayable image 40 for visually superimposing on the patient's tooth.

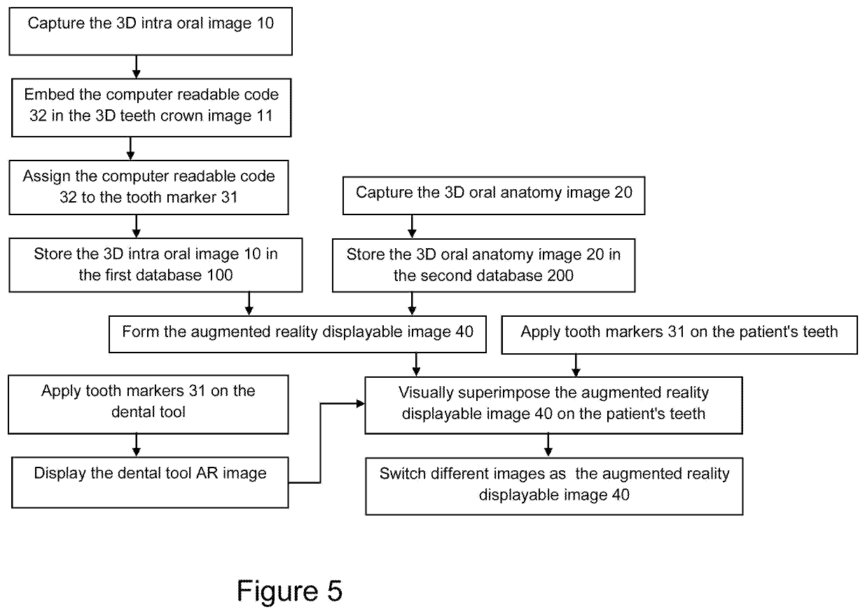

As shown in FIG. 5, the present invention further provides a method of overlaying teeth anatomy on a patient, which comprises the following steps.

(A) Store the 3D intra oral image 10 in the first database 100. Once the 3D teeth crown images 11 and the 3D gum image 12 are captured by the intra oral scanner, the 3D intra oral image 10 can be stored in the first database 100 which can be remotely stored in the cloud base server and/or the local server.

The step (A) further comprises the following steps.

(A.1) Embed the computer readable code 32 in the 3D teeth crown image 11 of the 3D intra oral image 10.

(A.2) Assign the computer readable code 32 to the tooth marker 31, so that when the tooth marker 31 is read, the augmented reality displayable image is retrieved for visually superimposing on the patient's teeth.

(B) Store the 3D oral anatomy image 20 in the second database 200. Once the 3D bone images 21 and the 3D nerve images 22 are captured by the dental CT scanner, the 3D oral anatomy image 20 can be stored in the second database 200 which can be remotely stored in the cloud base server and/or the local server.

(C) Form the augmented reality displayable image 40 by combining the 3D intra oral image 10 with the 3D oral anatomy image 20 via the processor 400.

(D) Apply one or more tooth markers 31 on one or more of the patient's teeth for tooth identification. Before the dental surgery, the tooth marker 31 can be directly applied on the exterior surface of the tooth to be treated for tooth identification.

(E) Visually superimpose the augmented reality displayable image 40 on the patient's teeth during the dental surgery when the tooth marker 31 is read by the augmented reality headset.

(F) If necessary, switch the 3D teeth crown image 10, the 3D gum image 12, the 3D bone image 21, and the 3D nerve image 22 as the augmented reality displayable image 40 for visually superimposing on the patient's tooth. It is preferred that the images are switched via the voice input from the augmented reality headset.

It is worth mentioning that the augmented reality headset can be the “Microsoft HoloLens” or the “Magic Leap AR device”. Because the operator knows the dimensions of the dental drill that will also have the tooth marker 31 and the computer readable code 32 of the teeth, not only will the operator know where the nerve and bone are, but also how deep they are drilling.

The present invention can apply to different dental surgeries. In one example, the operator will wear the augmented reality and will perform the implant or sinus surgery with the overlay of nerve, bone and sinus locations by overlaying the augmented reality displayable image 40 in the patient's mouth. The operator is able to perform the crown surgery by drilling on the tooth for a crown knowing where the nerve is as well as the position of the nerve. Root canals can be performed knowing the length of the tooth and location of the nerves as well as the location of the pulp chamber as marked in the 3D oral anatomy image 20. The operator can also perform sinus lift surgery or implant surgery knowing where the sinus is from the augmented reality displayable image 40 in view of the 3D oral anatomy image 20.

While the embodiments and alternatives of the invention have been shown and described, it will be apparent to one skilled in the art that various other changes and modifications can be made without departing from the spirit and scope of the invention.

What is claimed is:

1. An augmented reality dental display system, comprising:
a first database containing a 3D intra oral image which is constructed to have a plurality of 3D teeth crown images for showing crown portions of patient's teeth and a 3D gum image for showing a gum of the patient;
a second database containing a 3D oral anatomy image which is constructed to have a plurality of 3D bone images for showing a jaw bone and root portions of the teeth of the patient and a plurality of 3D nerve images for showing nerves of the patient;
a tooth identifier which comprises one or more tooth markers configured for applying on one or more of the patient's teeth for tooth identification, wherein the tooth marker is a readable code for an augmented reality headset; and
a processor combining the 3D intra oral image with the 3D oral anatomy image to form an augmented reality displayable image for visually superimposing on the patient's teeth during a dental surgery when the tooth marker is read by the augmented reality headset.

2. The augmented reality dental display system of claim 1, wherein the tooth marker is a fluorescence dye adapted for directly applying on an exterior surface of the patient's tooth.

3. The augmented reality dental display system of claim 1, wherein the tooth marker is in form of a QR code marker adapted for directly applying on an exterior surface of the patient's tooth.

4. The augmented reality dental display system of claim 2, wherein the tooth marker is in form of a QR code marker adapted for directly applying on the exterior surface of the patient's tooth.

5. The augmented reality dental display system of claim 1, wherein the tooth identifier further comprises one or more computer readable codes embedded in one or more of the 3D teeth crown images of the 3D intra oral image and assigned to one or more of the tooth markers, so that when the tooth marker is read, the augmented reality displayable image is retrieved for visually superimposing on the patient's teeth.

6. The augmented reality dental display system of claim 4, wherein the tooth identifier further comprises one or more computer readable codes embedded in one or more of the 3D teeth crown images of the 3D intra oral image and assigned to one or more of the tooth markers, so that when the tooth marker is read, the augmented reality displayable image is retrieved for visually superimposing on the patient's teeth.

7. The augmented reality dental display system of claim 6, wherein the computer readable code is a QR code being set corresponding to the QR code marker.

8. The augmented reality dental display system of claim 1, wherein the 3D nerve images contain nerves in bone images and nerves in tooth images.

9. The augmented reality dental display system of claim 7, wherein the 3D nerve images contain nerves in bone images and nerves in tooth images.

10. The augmented reality dental display system of claim 1, wherein when said tooth identifier is read for identifying the patient tooth, one or more of the 3D teeth crown image, the 3D gum image, the 3D bone image, and the 3D nerve image are switchably selected as the augmented reality displayable image for visually superimposing on the patient's tooth.

11. The augmented reality dental display system of claim 9, wherein when said tooth identifier is read for identifying the patient tooth, one or more of the 3D teeth crown image, the 3D gum image, the 3D bone image, and the 3D nerve image are switchably selected as the augmented reality displayable image for visually superimposing on the patient's tooth.

12. The augmented reality dental display system of claim 1, further comprising a voice command module operatively linked to the processor, wherein, in response to a voice input from the augmented reality headset to the voice command module, the processor is activated to selectively retrieve one or more of the 3D teeth crown image, the 3D gum image, the 3D bone image, and the 3D nerve image as the augmented reality displayable image for visually superimposing on the patient's tooth.

13. The augmented reality dental display system of claim 11, further comprising a voice command module operatively linked to the processor, wherein, in response to a voice input from the augmented reality headset to the voice command module, the processor is activated to selectively retrieve one or more of the 3D teeth crown image, the 3D gum image, the 3D bone image, and the 3D nerve image as the augmented reality displayable image for visually superimposing on the patient's tooth.

14. The augmented reality dental display system of claim 1, further comprising a cloud based server that remotely stores the first database and the second database.

15. The augmented reality dental display system of claim 13, further comprising a cloud based server that remotely stores the first database and the second database.

16. The augmented reality dental display system of claim 15, wherein the processor is configured to modify the augmented reality displayable image to change colors, to zoom in-and-out, to 3-dimensional rotate thereof.

17. The augmented reality dental display system of claim 15, further comprising a patient profile storing patient personal information and linking to the corresponding 3D intra oral image and 3D oral anatomy image in the first database and the second database respectively, wherein the patient profile is automatically retrieved to display the augmented reality displayable image when the tooth identifier is a read by the augmented reality headset.

18. A method of overlaying teeth anatomy on a patient, comprising the steps of:
(a) storing a 3D intra oral image in a first database, wherein the 3D intra oral image is constructed to have a plurality of 3D teeth crown images for showing crown portions of patient's teeth and a 3D gum image for showing a gum of the patient;
(b) storing a 3D oral anatomy image in a second database, wherein the 3D oral anatomy image is constructed to have a plurality of 3D bone images for showing a jaw bone and root portions of the teeth of the patient and a plurality of 3D nerve images for showing nerves of the patient;
(c) forming an augmented reality displayable image by combining the 3D intra oral image with the 3D oral anatomy image via a processor;
(d) applying one or more tooth markers on one or more of the patient's teeth for tooth identification, wherein the tooth marker is a readable code for an augmented reality headset; and
(f) visually superimposing the augmented reality displayable image on the patient's teeth during a dental surgery when the tooth marker is read by the augmented reality headset.

19. The method of claim 18, wherein the tooth marker is a fluorescence dye adapted for directly applying on an exterior surface of the patient's tooth.

20. The method of claim 19, wherein the 3D intra oral image is captured by an intra oral scanner to record the crown portions of the patient's teeth and to obtain a 3D visual crown information thereof.

21. The method of claim 20, wherein the 3D oral anatomy image is captured by dental CT scanner to record bone and nerve anatomy images with nerve in bone and nerve in tooth images.

22. The method of claim 21, wherein the step (a) further comprises the steps of:
(a.1) embedding one or more computer readable codes in one or more of the 3D teeth crown images of the 3D intra oral image; and
(a.2) assigning one or more of the computer readable codes to one or more of the tooth markers, so that when the tooth marker is read, the augmented reality displayable image is retrieved for visually superimposing on the patient's teeth.

23. The method of claim 22, wherein the tooth marker is in form of a QR code marker adapted for directly applying on the exterior surface of the patient's tooth, wherein the computer readable code is a QR code being set corresponding to the QR code marker.

24. The method of claim 23, wherein the step (f) further comprises a step of:
switchably selecting one or more of the 3D teeth crown image, the 3D gum image, the 3D bone image, and the 3D nerve image as the augmented reality displayable image for visually superimposing on the patient's tooth.

25. The method of claim 23, wherein the step (f) further comprises a step of:
switching the 3D teeth crown image, the 3D gum image, the 3D bone image, and the 3D nerve image as the augmented reality displayable image for visually superimposing on the patient's tooth via a voice input from the augmented reality headset.

26. The method of claim 23, further comprising a step of remotely storing the first database and the second database in a cloud based server.
